SEQUENCE LISTING (5 sequences)

SEQ ID NO: 1 - protein, 8 aa, Artificial Sequence
  synthetic sequence tag for labeling proteins
DYKDDDDK

SEQ ID NO: 2 - protein, 6 aa, Artificial Sequence
  synthetic sequence tag for labeling proteins
HHHHHH

SEQ ID NO: 3 - protein, 10 aa, Artificial Sequence
  synthetic sequence tag for labeling proteins
ILKKATAYIL

SEQ ID NO: 4 - protein, 10 aa, Artificial Sequence
  synthetic sequence tag for labeling proteins
EQKLISEEDL

SEQ ID NO: 5 - protein, 6 aa, Artificial Sequence
  synthetic sequence tag for labeling proteins
YPYDVP

(19) United States
(12) Patent Application Publication
(10) Pub. No.: US 2020/0237923 A1
(43) Pub. Date: Jul. 30, 2020

(54) PROTEIN BIOCONJUGATION METHOD

(71) Applicant: THE JOHNS HOPKINS UNIVERSITY, Baltimore, MD (US)

(72) Inventors: Bert Vogelstein, Baltimore, MD (US); Kenneth W. Kinzler, Baltimore, MD (US); Shibin Zhou, Owings Mills, MD (US); Surojit Sur, Gaithersburg, MD (US)

(21) Appl. No.: 15/768,053

(22) PCT Filed: Oct. 14, 2016
(86) PCT No.: PCT/US2016/056981

§ 371 Date: Apr. 13, 2018

Related U.S. Application Data
(60) Provisional application No. 62/241,378, filed on Oct. 14, 2015.
(60) Provisional application No. 62/293,001, filed on Feb. 9, 2016.

(51) Int. Cl. A61K 47/60; C07K 1/107; C12P 21/06

(52) U.S. Cl. CPC A61K 47/60; C12Y 304/00; C12P 21/06; C07K 1/1077

(57) ABSTRACT

Chemical conjugation is commonly used to enhance the pharmacokinetics, biodistribution, and potency of protein therapeutics, but often leads to non-specific modification or loss of bioactivity. Here, we present a simple, versatile and widely applicable method that allows exquisite N-terminal specific modification of proteins. Combining reversible side-chain blocking and protease mediated cleavage of a commonly used HIS tag appended to a protein, we generate with high yield and purity exquisitely site specific and selective bio-conjugates of TNF-α by using amine reactive NHS ester chemistry. We confirm the N terminal selectivity and specificity using mass spectral analyses and show near complete retention of the biological activity of our model protein both in vitro and in vivo murine models. This methodology is applicable to a variety of potentially therapeutic proteins and the specificity afforded by this technique allows for rapid generation of novel biologics.

### CROSS-REFERENCE TO RELATED APPLICATIONS

This application claims the benefit of U.S. Provisional Application No. 62/293,001, filed Feb. 9, 2016, and U.S. Provisional Application No. 62/241,378, filed Oct. 14, 2015, each of which is incorporated herein by reference in its entirety.

### STATEMENT OF GOVERNMENTAL INTEREST

This invention was made with government support under grant nos. CA 43460, CA 57345, and CA 62924, awarded by the National Institutes of Health. The government has certain rights in the invention.

### FIELD OF THE INVENTION

This invention is related to the area of protein chemistry. In particular, it relates to modification of proteins, polypeptides, and peptides.

### INCORPORATION-BY-REFERENCE OF MATERIAL SUBMITTED ELECTRONICALLY

This application contains a sequence listing. It has been submitted electronically via EFS-Web as an ASCII text file entitled “P13837-03_ST25.txt.” The sequence listing is 1,315 bytes in size, and was created on Oct. 14, 2016. It is hereby incorporated by reference in its entirety.

### BACKGROUND OF THE INVENTION

The use of proteins and peptides for therapeutic applications are often compromised by low biological stability, high renal clearance, and non-optimal biodistribution^{1,2}. Chemical attachment of poly-(ethylene glycol) (PEGylation) is often considered the most effective way to improve these pharmacologic properties by increasing circulation half-life, reduce the immunogenicity of proteins and protease mediated degradation³⁻⁶. However, random conjugation results in heterogeneous derivatives with undefined composition and can substantially lower the bioactivity of the modified protein, leading to unpredictable in vivo behavior. The same issues apply to conjugations for other purposes, such as the attachment of toxic small molecules to increase the therapeutic efficacy of antibodies.

Site-specific modification of proteins is therefore an attractive approach to circumvent the non-specificity resulting from random conjugation to amines, thiol, or other specific amino acids on proteins. Currently used site-specific strategies exploit rare chemoselective anchors present either naturally or introduced artificially into protein backbones⁷. Amino terminal serines or threonines can be oxidized to aldehydes and targeted using aldehyde-reactive PEG reagents⁸⁻¹¹, cysteines have been targeted using thiol-reactive agents¹²⁻¹⁵, and in a few cases the pKa difference between the α and the ϵNH2 groups have been used successfully¹⁶⁻¹⁸. Attempts have even been made to replace all internal lysines to achieve N-terminal selective conjugations^{19,20}. A recent report has shown that 2-pyridinecarboxaldehydes react with the N terminus of proteins resulting in the formation of imidazolidinone bound conjugates²¹. All of these techniques can be usefully employed, but in view of the ubiquity of this problem and its importance, new ways to site-specifically modify proteins, regardless of the tag used for purification, and with inexpensive, commercially available reagents, are still a high priority.

### SUMMARY OF THE INVENTION

According to one aspect of the invention a method of modifying the N-terminus or the C-terminus of a peptide, polypeptide, or protein is provided. A derivative of the peptide, polypeptide, or protein is incubated in the presence of a reversible amine group blocking agent so that all amine groups in the derivative are blocked or in the presence of a reversible carboxyl group blocking agent so that all carboxyl groups in the derivative are blocked, wherein the derivative comprises an amino acid tag and a protease cleavage site appended to the N-terminus or the C-terminus of the peptide, polypeptide, or protein, such that the protease cleavage site is interposed between the amino acid tag and the N-terminus or the C-terminus of the peptide, polypeptide, or protein. The blocked derivative is contacted with a protease that specifically cleaves at the protease cleavage site whereby the blocked derivative is cleaved. The cleaved derivative is incubated with an amine reactive form of a reagent in a reaction mixture, whereby the N-terminus of the cleaved derivative is modified with the reagent to form a reagent-esterified, cleaved derivative or incubating the cleaved derivative with a carboxyl reactive form of a reagent in a reaction mixture, whereby the C-terminus of the cleaved derivative is modified with the reagent to form a reagent-esterified, cleaved derivative. Blocking groups are removed from the amine groups or from the carboxyl groups in the reagent-esterified cleaved derivative.

According to another aspect of the invention a preparation is provided. The preparation is a bioactive peptide, polypeptide, or protein that is modified at its N-terminus or its C-terminus by esterification with a reagent. The preparation is homogeneous in the location of the esterification on the peptide, polypeptide, or protein. And the bioactivity of the modified peptide, polypeptide, or protein is equivalent to the bioactivity of the peptide, polypeptide, or protein without modification.

According to another aspect of the invention a kit for modifying a peptide, polypeptide, or protein is provided. The kit comprises a reversible amine blocking agent or a reversible carboxyl blocking agent, and a protease.

These and other embodiments which will be apparent to those of skill in the art upon reading the specification provide the art with

### BRIEF DESCRIPTION OF THE DRAWINGS

FIG. 1: Schematic representation of PRINT PEGylation. The reaction proceeds through blockage of reactive side chains (II), followed by protease mediated cleavage to reveal a single reaction site at the N terminus (III). Conjugation with NHS ester and subsequent deprotection of side chains leads to N terminal selective and specific conjugate (IV). Direct conjugation of the protein using the same NHS ester leads to heterogeneous population of conjugates (V).

FIG. 2A-2B: FIG. 2A. SDS-PAGE characterization of scTNF-α derivatives: Lanes (left to right): Protein standard; Lane 1, His tagged scTNF-α (I); Lane 2, cleaved scTNF-α (scTNF-α) (II); Lane 3, directly PEGylated PEGSK scTNF-α (random PEGSK scTNF-α) (V); Lane 4, PRINT PEGSK scTNF-α (IV). FIG. 2B. SEC HPLC of PRINT PEGSK scTNF-α.0

FIG. 3A-3C: FIG. 3A. In vitro bioactivity of scTNF-α derivatives in L929 cells. FIG. 3B. In vitro serum stability and residual activity of scTNF-α, PRINT PEGSK and PRINT PEG2OK scTNF-α. FIG. 3C. In vivo clearance of scTNF-α and its PEGylated derivatives.

FIG. 4: List of conjugating reagents.

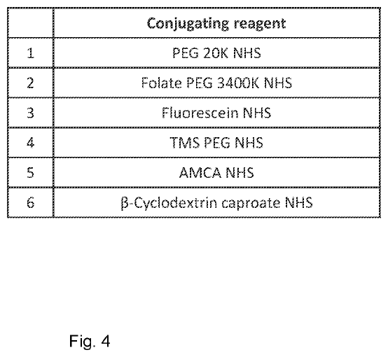

FIG. 5A-5B: FIG. 5A. SDS PAGE characterization: Lanes from left to right: protein standard, Lane 1, His tagged scTNF-α; Lane 2, CA protected protease cleaved scTNF-α; Lane 3, CA protected His-tagged scTNF-α treated with 1000×PEG 5K NHS; Lane 4, PRINT Fluorescein scTNF-α; Lane 5, PRINT PEGSK scTNF-α; Lane 6, random PEGSK scTNF-α; Lane 7, PRINT PEG2OK scTNF-α. FIG. 5B. Overlay of SEC-HPLC of Fluorescein scTNF at two wavelengths 220 (black) nm and 482 nm (green).

FIG. 6A-6B: FIG. 6A. MS/MS analyses of the tryptic peptide GRSSQNSSDKPVAH modified with Fluorescein: Fluorescein NHS ester was used instead of PEGSK NHS, allowing us to identify peptide fragments labelled with an exact mass of 358.04 (arrows). The b ions are shown in red, y ions are shown in blue. Fragment ion masses were consistent with modification at the N-terminus and no other peptides with a mass increase of 358.04 were detected. FIG. 6B. List of peptides with additional mass of 358, detected for tryptic fragment GRSSQNSSDKPVAH.

FIG. 7: Acute toxicity of wt TNF-α, scTNF-α and its derivatives in BALB/c mice harboring CT26 tumors. Ten mice in each study arm were injected with a single i.v. dose of various forms of TNF-α at different doses. The results were scored by surviving mice at the end of a 24 h time period.

FIG. 8: Additional bioconjugates made using scTNF-α and the listed NHS-reagent have been completely characterized. Additional bioconjugates have been made using GFP as well as Ferritin.

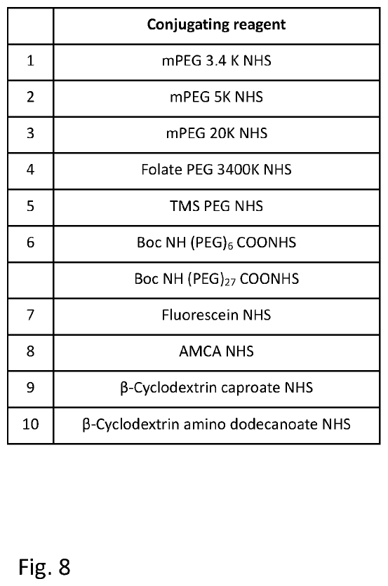

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

The inventors have developed a novel technique named PRINT (PRotect, INcise Tag) for N-terminal specific bioconjugation of proteins and peptides. In particular embodiments, PRINT can be used for selective attachment of any desired entity bearing a nitrogen-reactive functionality. In specific embodiments, we show that PRINT is able to engineer exclusive N-terminal conjugation of a model protein without altering its biological properties. In alternative embodiments, the same principles of PRINT apply for C-terminal specific bioconjugation, except that the desired entity bears a carboxyl-reactive functionality. PRINT is one particular embodiment within a much broader class of reactions schemes that applies to different blocking groups and different reactive chemistries.

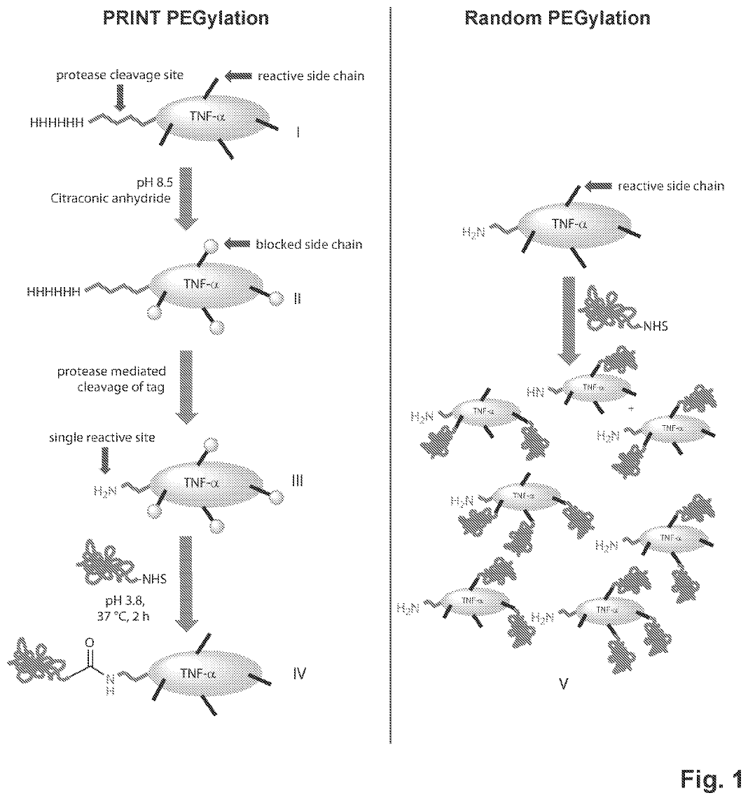

PRINT Design. PRINT was conceptualized to enable N-terminal specific chemical modification, while traditional chemical modification of proteins using amine-reactive NHS ester chemistry leads to heterogeneous and multiple modifications on internal reactive ϵNH2 groups (FIG. 1). PRINT can be used on any protein that has any desired N-terminal tag (to enhance purification) and any protease cleavage site (to eradicate the tag prior to final purification). (FIG. 1, I). The recombinant protein is first treated with an excess of citraconic anhydride to reversibly block all reactive primary amine sites (FIG. 1, II). Proteolytic cleavage will then expose only a single amine (the a primary amine at the N-terminus) for desired bioconjugation by amine-reactive NHS ester chemistry (FIG. 1, III). Lowering of reaction pH will result in removal of the citraconates, leaving homogeneous protein molecules modified at the N-terminus (FIG. 1, IV).

As proof of principle, we used Tumor Necrosis Factor-α (TNF-α) to demonstrate the efficiency and specificity of PRINT. A well characterized cytokine, TNF-α has gained attention as a vascular-disrupting agent specific to tumors²²⁻²⁵. However, TNF-α, like many other potential therapeutic proteins, suffers from inherent instability and short biological half-life, and exhibits toxic side effects at therapeutic concentrations in both small animals and human patients. Altering its pharmacokinetic profile by PEGylation has been shown to enhance its stability and bioavailability, and to mitigate its toxicity^{19,20,26-28}. In this study, we used a recombinant single-chain form consisting of three head-to-tail copies of the monomer, as this has been shown to enhance formation of an active protein from bacteria²⁹.

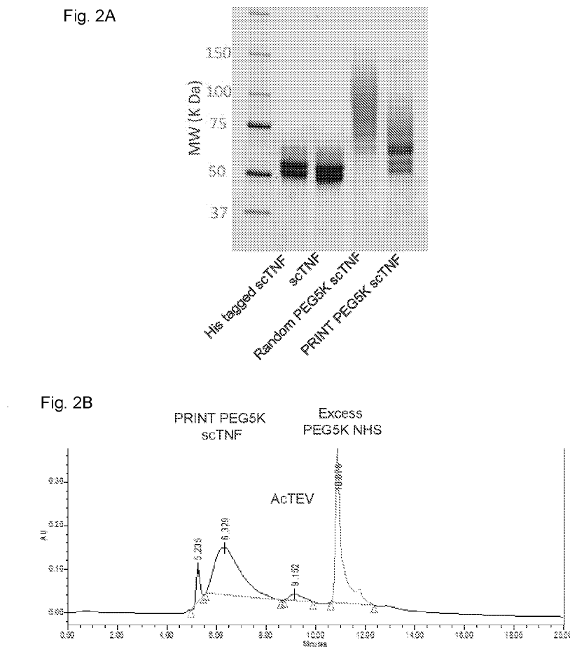

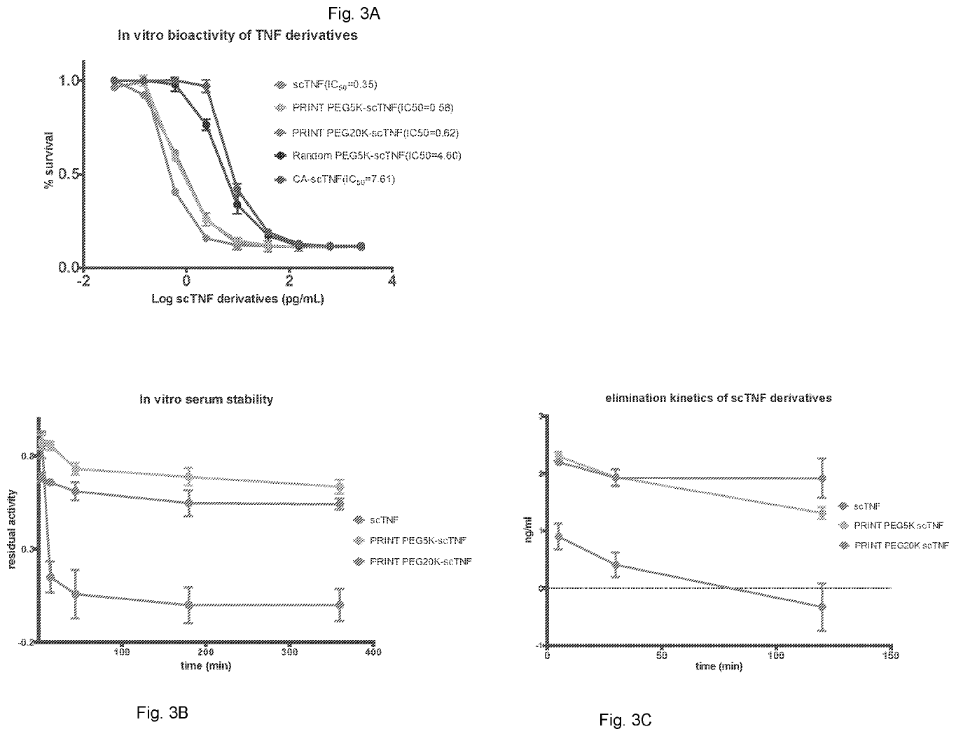

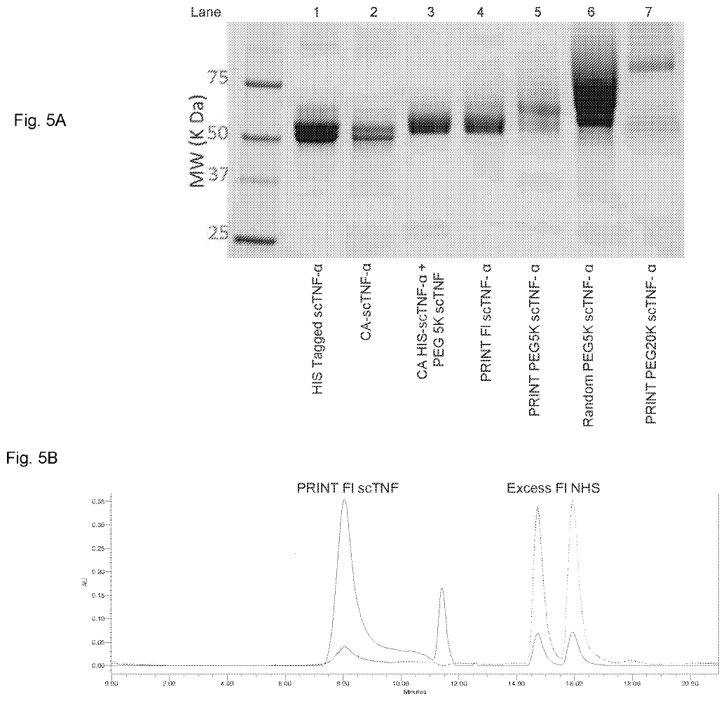

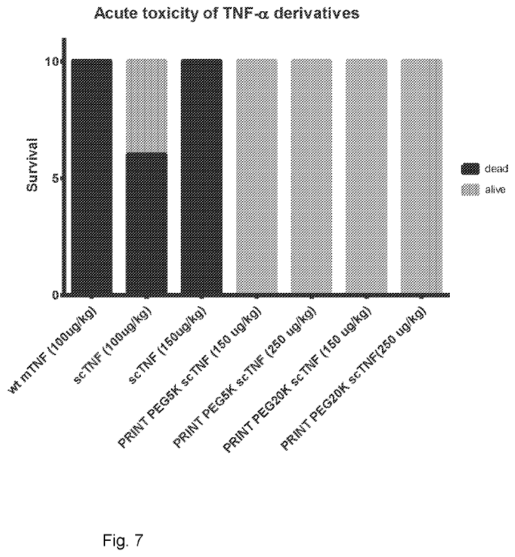

As shown below in the examples, we have demonstrated that the side chain protection before cleavage of the tag efficiently blocked all reactions at the side chains (FIG. 5A, Lane 3). The single product formed after protease-mediated tag removal and N-terminal conjugation suggests exquisite selectivity and specificity in contrast to conventional reaction using the same NHS reagent (compare FIG. 2A Lane 4, FIG. 5A Lanes 4, 5 and 7 with FIG. 1A, Lane 3 and FIG. 5A, Lane 6), which was further confirmed by mass spectrometric analyses. Subsequent de-blocking generated an N-terminal protected TNF-α molecule with enhanced serum stability, superior pharmacokinetic properties, and reduced systemic toxicity (FIG. 3B-C and FIG. 7). Importantly, N-terminal protection by PRINT did not affect the bioactivity of TNF-α (FIG. 3A).

As noted in the background of the invention, existing site-selective bioconjugation approaches are either specific to amino acid tags^{7-11,30,31} or involve substantial non-trivial chemical^{18,21} or biotechnological manipulations^{19,20} to synthesize a desired bioconjugate. In contrast, PRINT employs ubiquitously used recombinant DNA techniques and easily acquired commercial reagents to generate exquisite N-terminal selective protection. In this study, we used TNF-α as an example to show that PRINT is a robust, reproducible and mild strategy which is able to target the α-amine and provide N-terminal specific protection to proteins or peptides that suffer from similar issues. In other embodiments, PRINT can be used to generate a variety of N-terminal conjugates using NHS ester chemistry on any recombinant protein or peptide bearing a cleavable purification tag. We believe that this approach is strongly orthogonal to current methods and will be applicable to many biotherapeutics and bioprobes that are currently being designed to treat cancer or other diseases.

Reactive amine reagents can be any known in the art, including but not limited to active esters and carboxylic acids, succinimidyl esters such as NHS, tetrafluorophenyl (TFP) Esters, Sulfodichlorophenol (SDP) Esters, aldehydes, carbonyl azides, sulfonyl chlorides, FITC, and isothiocyanates.

Reactive carboxyl reagents can be any known in the art, including but not limited to hydrazines, hydroxylamine, amines, aliphatic amine derivatives and fluorescent trifluoromethanesulfonate.

Reversible blocking agents for amine groups include maleic anhydride, methylmaleic anhydride, sulfo-NHS-acetate, citraconic anhydride, and TFCS. Any can be used as is convenient.

Reversible blocking agents for carboxyl groups include t-butyloxycarbonyl azide

(BOC azide), diazomethane, and phenyldiazomethane. Any can be used as is convenient.

Amino acid tags which may be used are any that are known in the art. These include without limitation, FLAG tags, e.g., N-DYKDDDDK-C (SEQ ID NO: 1), polyhistidine tags (e.g., (HHHHHH) (SEQ ID NO: 2)), MYC tags, e.g., N-ILKKATAYIL-C (SEQ ID NO: 3), and N-EQKLISEEDL-C (SEQ ID NO: 4), HA tags, e.g., N-YPYDVP-C (SEQ ID NO: 5).

Proteases which can be used in the invention are any that are site specific and which preferably do not have a cleavage site within the peptide, polypeptide, or protein. Suitable proteases include TEV endoprotease, Factor X, and thrombin, to name just a few.

Kits comprise a package that is either divided or undivided. Typically each individual element or reagent is provided in a separate vessel. Instructions may be included, optionally. The kits may comprise a reversible amine blocking agent or a reversible carboxyl blocking agent and/or a protease. The kits may comprise a first buffer suitable for the reversible amine blocking agent to block free amine groups and a second buffer suitable for removal of blocking groups from the amine groups. Alternatively, the kit may comprise a first buffer suitable for the reversible carboxyl blocking agent to block free carboxyl groups, and a second buffer suitable for removal of blocking groups from the carboxyl groups.

Reagents for use in the method are either amine reactive forms or carboxyl reactive forms. The reagent may be any desired functionality to be added to the peptide, polypeptide, or protein. The reagent may be a peptide, a polypeptide, a cytotoxic agent, a ligand which specifically binds to a receptor, an antibody, an antibody fragment, a cytokine, a growth factor, a blood clotting factor, an imaging contrast agent, a radionuclide, a fluorescent moiety, a biopolymer, polyethylene glycol, β-Cyclodextrin caproate, β-Cyclodextrin amino dodecanoate, any cyclodextrin α, β or γ, or any cavitand with a suitable linker.

The peptide, polypeptide, or protein which is modified by the method may be any that is of interest. It may be, for example, an antibody, an antibody fragment, such as an ScFv, a cytotoxic agent, TNF-α, a cytokine, a growth factor, a blood clotting factor. Any peptide, polypeptide, or protein may be used without limitation.

Reversible blocking may be pH dependent. Other means of reversible blocking as are known in the art may be used as well.

Properties of the modified peptide will preferably be improved in some aspect. Aspects which may be improved include without limitation: serum stability, pharmacokinetic properties, biodistribution, renal clearance, systemic toxicity, molecular or cellular targeting, and/or imaging contrast.

The overall scheme provides the ability to join two proteins or a protein and another entity without use of fusion protein expression. Such expression often leads to functional loss due to misfolding. Although the scheme requires production of an amino acid tagged protein, typically by recombinant expression, no loss of protein function has been observed to date.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example 1—Materials and Methods

General Materials and Methods: Citraconic anhydride(Sigma), Sodium phosphate dibasic and monobasic (Sigma), mPEG 5K NHS ester (NANOCS), mPEG 20K NHS ester (NANOCS), Fluorescein NHS ester (NANOCS) and AcTEV (Life Technologies) were obtained from commercial sources and used as is. Single chain TNF-α (scTNF) was designed according to a published sequence and the recombinant protein was produced by GeneArt in HEK293 mammalian expression system. All animal experiments were designed in accordance with the National Institute of Health's Guide for the Care and Use of Laboratory Animals and were approved by The Johns Hopkins University's Institutional Animal Care and Use Committee.

Direct Conjugation: scTNF-α (1 mg/ml in PBS) was treated with PEG NHS ester (1 mg) for 1 h at room temperature and excess reagents were removed by dialyses. The recovered product was analyzed and quantitation by done by SDS-PAGE and used as such for in vitro and in vivo animal experiments.

PRINT Conjugation: scTNF-α (1 mg/ml in 200 mM phosphate buffer at pH 8.5) was treated with citraconic anhydride (3 ul/100 ug protein) at room temperature32 for 5 minutes. The mixture was then dialyzed against 500 ml phosphate buffer (200 mM, pH 8.5) for 8 hours. AcTEV (5 ul/100 ug protein) was then added and the mixture allowed to shake gently at room temp overnight. PEG NHS esters (20-50×) was then added and the mixture allowed to incubate for 1 hour at room temp. The mixture was then dialyzed against 1 L acetate buffer (200 mM, pH 3.8) at room temperature overnight followed by buffer exchange against PBS 1 L twice. AcTEV was then removed from the product by NiNTA spin columns following manufacturer instructions. The products were then analyzed for purity and quantitated for protein content by SDS-PAGE and used as such for in vitro and in vivo animal experiments. For Mass Spectral analyses, the product was further purified by Size Exclusion chromatography using a Phenomenex BioSep-SEC-s2000 (300×7.8 mm) column. Samples of 100 ul were injected, and separations carried out using PBS (pH 7.4) as the mobile phase at ambient temperature and flow rate of 1.00 ml/min on a Waters D600 HPLC system using Absorbance at 220 nm.

SDS-PAGE and protein quantitation: Protein samples were analyzed for purity using Biorad Stain Free TGX precast gels. In brief, 3 ul of protein samples was diluted with deionized water (6 ul) followed by 3 ul of Laemlli buffer (4×). After electrophoresis, gels was developed using a Biorad ChemiDoc MP imaging system and quantitation was performed using Imagelab software against standards containing known quantity of scTNF-α.

Mass Spectral Analyses by Liquid Chromatography-Tandem Mass Spectrometry (LC-MS): Protein samples from either gel bands or size-exclusion chromatography were proteolyzed with trypsin as described previously. Digested peptides were extracted and subjected to vacuum drying in a Speedvac followed by reconstitution in 5 μL of 2% acetonitrile/0.1% formic acid for further analysis by liquid chromatography/tandem mass spectrometry (LC-MS/MS) using LTQ Orbitrap Velos (2) MS (Thermo Fisher Scientific). For data analysis the data was submitted for a Sequest search using Proteome Discoverer v 1.3 (Thermo Fisher Scientific) against the constructed sequence database. The Fluorescein modification of 358.040 was set to variable at K and Y and static for the N-terminus.

In vitro cytotoxicity assay: Conjugated proteins were assessed for bioactivity using previously described TNF-α induced killing of L929 cells. L929 cells (Sigma #85011425) were plated at density of 3.5×105 cells per well in 96 well plates and incubated overnight at 37° C. in a humidified incubator. A 4 fold dilution series for each sample was created starting at 2.5 ng/mL. Cells were then treated with 50 ul of TNF derivatives at each concentration along with 50 ul Actinomycin D (4 ug /ml) and allowed to incubate 24 h. Potency of the TNF-α derivatives was assayed using cell proliferation reagent WST-1 (Roche Lifesciences) following manufacturers protocol.

In vitro stability assay: scTNF-α and its PRINT Pegylated derivatives were incubated with mouse serum at 37° C. for 24 h and aliquots were collected at various time points (5, 15, 45 min, 1.5, 3, 6 and 12 h) and frozen immediately. Once all desired time points were collected, the samples were thawed and analyzed for residual bioactivity using the L929 cytotoxicity assay.

In vivo pharmacokinetics: The pharmacokinetic characteristics of scTNF-α derivatives was investigated in mice following intravenous (i.v.) administration. Healthy female BALB/c mice were randomly divided to 3 groups (n=3) and each group was administered 150 μg/kg (protein base) of TNF-α erivatives Blood samples were collected at different time points (5, 30 min and 2 h) after i.v. injection, and plasma were obtained by centrifugation and stored at −70° C. until required for the assay. scTNF-α concentrations in mice plasma were measured and quantitated using a commercial TNF ELISA kit (R & D Systems) and a dilution series of known amounts of scTNF-α as standard.

Acknowledgments: We would like to thank Evangeline Watson for expert technical assistance with animal experiments, and Evan Brower, Kibem Kim, Ashley Cook and Margaret Hoang for helpful comments and discussions. This project was supported by the Virginia and D. K. Ludwig Fund for Cancer Research and grants CA062924 and CA 043460 from the National Institutes of Health.

### Example 2

PRINT using scTNF-α as a model protein. A recombinant single-chain TNF-α (scTNF-α) containing a His-tag and TEV protease cleavage site was designed based on a published sequence29. After affinity purification through a nickel-nitrilotriacetic acid (Ni-NTA) column, the His-tagged scTNF-a was treated with a 1000-fold molar excess of citraconic anhydride. Excess reagent was removed by dialysis and the citraconylated protein was subjected to overnight digestion with AcTEV protease. After complete proteolytic cleavage of the His-tag, NHS ester of PEG-5000 (PEGSK) was added and the mixture allowed to shake at room temperature for 30 minutes. Excess reagent was then removed and pH adjusted to 3.8 for deprotection of side chains. These treatments yielded a major N-terminal mono PEGylated species (FIG. 2A Lane 4). In comparison, a traditional PEGylation method without PRINT generated multiple species of various lengths, indicating the expected large and variable numbers of internal reactive NH2 groups getting PEGylated (FIG. 2A Lane 3). A control PEGylation on citraconylated scTNF prior to removal of its His-tag yielded no PEGylated products (FIG. 5A Lane 3), demonstrating complete blocking of the reactive α and ϵNH2 groups present on the protein. Because this process was so simple and effective, several other conjugates of scTNF-α were able to be synthesized for biological evaluation starting from small amounts of purified proteins.

### Example 3

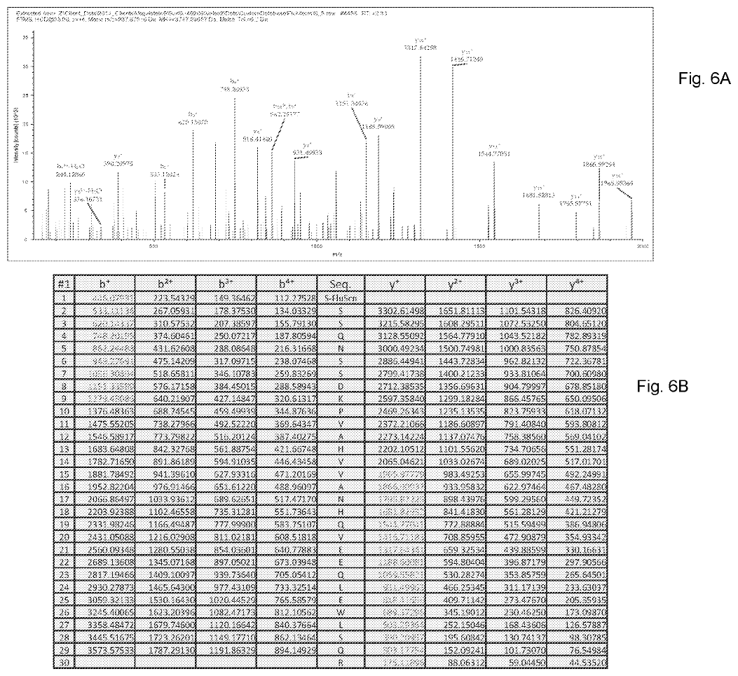

PRINT provides N terminal selectivity. To elucidate the exact location of the conjugation, we replaced the reactive PEGSK with fluorescein NHS (Fl) ester, a smaller adduct with a known exact mass of 358 Da (FIG. 5A, lane 4 and FIG. 5B). Size exclusion high-performance liquid chromatography (HPLC) analysis of PRINT PEGylated scTNF-α revealed the formation of a single major product (FIG. 2B). Proteolytic cleavage of PRINT flourescein scTNF-α with trypsin followed by mass spectral analysis confirmed the presence of a single fluorescein molecule at the N-terminal serine (FIG. 6A). No other peptide fragment containing fluorescein was detected (FIG. 6B), suggesting an exquisite N-terminal selectivity and specificity of the reaction.

### Example 4

PRINT retains bioactivity of scTNF-α. To assess bioactivity of the PRINT PEGylated scTNF-α, we performed a cytotoxicity assay using L929 cells that express TNFR1, the receptor mediating TNF-α induced cytotoxicity. Unmodified scTNF-α and scTNF-α that had been PRINT-PEGylated with PEGSK or PEG-20000 (PEG20K) all showed similar cytotoxic activity against L929 cells, with EC50 of 0.35, 0.58 and 0.62 pg/mL, respectively (FIG. 3A). In contrast, randomly PEGylated scTNF-α suffered more than ten-fold loss of activity, resulting in an EC50 of 4.6 pg/mL. Similarly, global blocking of lysine side chains by citraconylation dramatically reduced (EC50=7.6 pg/mL) its bioactivity, thereby providing biological confirmation that the citraconate groups had been removed.

### Example 5

PRINT reduces scTNF-α toxicity. To assess toxicity in vivo, wild-type mouse TNF-α, unmodified scTNF-α and PRINT-PEGylated (PEGSK) scTNF-α were intravenously injected at various doses into BALB/c mice bearing large subcutaneous CT26 tumors. Mice bearing large tumors were used because they are more sensitive to TNF-α induced toxicity than non-tumor-bearing mice (ref here). At a dose of 150 μg/kg all 10 animals treated with mouse wt TNF-α or unmodified scTNF-α died within 24 hours. In contrast, none of the 10 animals treated with PRINT PEGylated (PEGSK or PEG20K) scTNF-α at the same or higher doses showed any adverse event (FIG. 6A).

### Example 6

PRINT enhances stability and circulation half-life of scTNF-α. Finally, we evaluated stability of the unmodified scTNF-α, PRINT PEGylated (PEGSK) scTNF-α, and PRINT-PEGylated (PEG20K) scTNF-α. We first assessed their serum stability ex vivo. Both PRINT-PEGylated scTNF-α molecules showed greatly improved stability compared to the unmodified scTNF-α (FIG. 3b). We then intravenously injected the TNF-a preparations into non-tumor-bearing healthy BALB/c mice and collected blood samples at various time points. The unmodified scTNF-α showed a rapid clearance from the bloodstream, as assessed by enzyme-linked immunosorbent assay (ELISA), and was undetectable at 2 h (FIG. 3c). In contrast, the two PRINT PEGylated scTNF-α molecules showed substantially higher persistence in the bloodstream and low clearance rate.

### REFERENCES

The disclosure of each reference cited is expressly incorporated herein.

1 Krejsa, C., Rogge, M. & Sadee, W. Protein therapeutics: new applications for pharmacogenetics. Nature reviews. Drug discovery 5, 507-521, doi:10.1038/nrd2039 (2006).

2 Frokjaer, S. & Otzen, D. E. Protein drug stability: a formulation challenge. Nature reviews. Drug discovery 4, 298-306, doi:10.1038/nrd1695 (2005).

3 Harris, J. M. & Chess, R. B. Effect of pegylation on pharmaceuticals. Nature reviews. Drug discovery 2, 214-221, doi:10.1038/nrd1033 (2003).

4 Obermeier, B., Wurm, F., Mangold, C. & Frey, H. Multifunctional Poly(ethylene glycol)s. Angewandte Chemie 50, 7988-7997, doi:10.1002/anie.201100027 (2011).

5 Veronese, F. M. & Mero, A. The impact of PEGylation on biological therapies. BioDrugs : clinical immunotherapeutics, biopharmaceuticals and gene therapy 22, 315-329 (2008).

6 Jevsevar, S., Kunstelj, M. & Porekar, V. G. PEGylation of therapeutic proteins. Biotechnology journal 5, 113-128, doi:10.1002/biot.200900218 (2010).

7 Stephanopoulos, N. & Francis, M. B. Choosing an effective protein bioconjugation strategy. Nature chemical biology 7, 876-884, doi:10.1038/nchembio.720 (2011).

8 Rabuka, D., Rush, J. S., deHart, G. W., Wu, P. & Bertozzi, C. R. Site-specific chemical protein conjugation using genetically encoded aldehyde tags. Nature protocols 7, 1052-1067, doi:10.1038/nprot.2012.045 (2012).

9 Wu, P. et al. Site-specific chemical modification of recombinant proteins produced in mammalian cells by using the genetically encoded aldehyde tag. Proceedings of the National Academy of Sciences of the United States of America 106, 3000-3005, doi:10.1073/pnas.0807820106 (2009).

10 Frese, M. A. & Dierks, T. Formylglycine aldehyde Tag-protein engineering through a novel post-translational modification. Chembiochem : a European journal of chemical biology 10, 425-427, doi:10.1002/cbic.200800801 (2009).

11 Agarwal, P., van der Weijden, J., Sletten, E. M., Rabuka, D. & Bertozzi, C. R. A Pictet-Spengler ligation for protein chemical modification. Proceedings of the National Academy of Sciences of the United States of America 110, 46-51, doi:10.1073/pnas.1213186110 (2013).

12 Pan, L. Q. et al. Site-specific PEGylation of a mutated-cysteine residue and its effect on tumor necrosis factor (TNF)-related apoptosis-inducing ligand (TRAIL). Biomaterials 34, 9115-9123, doi:10.1016/j.biomaterials.2013.08.020 (2013).

13 Doherty, D. H. et al. Site-specific PEGylation of engineered cysteine analogues of recombinant human granulocyte-macrophage colony-stimulating factor. Bioconjugate chemistry 16, 1291-1298, doi:10.1021/bc050172r (2005).

14 Shaunak, S. et al. Site-specific PEGylation of native disulfide bonds in therapeutic proteins. Nature chemical biology 2, 312-313, doi:10.1038/nchembio786 (2006).

15 Brocchini, S. et al. Disulfide bridge based PEGylation of proteins. Advanced drug delivery reviews 60, 3-12, doi:10.1016/j.addr.2007.06.014 (2008).

16 Hao, Y., Chen, J., Wang, X., Zhu, H. & Rong, Z. Effects of site-specific polyethylene glycol modification of recombinant human granulocyte colony-stimulating factor on its biologic activities. BioDrugs : clinical immunotherapeutics, biopharmaceuticals and gene therapy 20, 357-362 (2006).

17 Baker, D. P. et al. N-terminally PEGylated human interferon-beta-1a with improved pharmacokinetic properties and in vivo efficacy in a melanoma angiogenesis model. Bioconjugate chemistry 17, 179-188, doi:10.1021/bc050237q (2006).

18 Chan, A. O. et al. Modification of N-terminal alpha-amino groups of peptides and proteins using ketenes. Journal of the American Chemical Society 134, 2589-2598, doi:10.1021/ja208009r (2012).

19 Yamamoto, Y. et al. Site-specific PEGylation of a lysine-deficient TNF-alpha with full bioactivity. Nature biotechnology 21, 546-552, doi:10.1038/nbt812 (2003).

20 Yoshioka, Y. et al. Optimal site-specific PEGylation of mutant TNF-alpha improves its antitumor potency. Biochemical and biophysical research communications 315, 808-814, doi:10.1016/j.bbrc.2004.01.125 (2004).

21 MacDonald, J. I., Munch, H. K., Moore, T. & Francis, M. B. One-step site-specific modification of native proteins with 2-pyridinecarboxyaldehydes. Nature chemical biology 11, 326-331, doi:10.1038/nchembio.1792 (2015).

22 Lu, L. et al. Vascular-targeted TNFalpha improves tumor blood vessel function and enhances antitumor immunity and chemotherapy in colorectal cancer. Journal of controlled release: official journal of the Controlled Release Society, doi:10.1016/j.jconre1.2015.05.282 (2015).

23 Lejeune, F. J. & Ruegg, C. Recombinant human tumor necrosis factor: an efficient agent for cancer treatment. Bulletin du cancer 93, E90-100 (2006).

24 Seynhaeve, A. L. et al. Tumor necrosis factor alpha mediates homogeneous distribution of liposomes in murine melanoma that contributes to a better tumor response. Cancer research 67, 9455-9462, doi:10.1158/0008-5472.CAN-07-1599 (2007).

25 Qiao, Y. et al. A robust approach to enhance tumor-selective accumulation of nanoparticles. Oncotarget 2, 59-68 (2011).

26 Li, Y. P. et al. PEGylated recombinant human tumor necrosis factor alpha: preparation and anti-tumor potency. Acta pharmacologica Sinica 22, 549-555 (2001).

27 Jiang, Y. Y., Liu, C., Hong, M. H., Zhu, S. J. & Pei, Y. Y. Tumor cell targeting of transferrin-PEG-TNF-alpha conjugate via a receptor-mediated delivery system: design, synthesis, and biological evaluation. Bioconjugate chemistry 18, 41-49, doi:10.1021/bc060135f (2007).

28 Thamm, D. H. et al. Preclinical investigation of PEGylated tumor necrosis factor alpha in dogs with spontaneous tumors: phase I evaluation. Clinical cancer research: an official journal of the American Association for Cancer Research 16, 1498-1508, doi:10.1158/1078-0432.CCR-09-2804 (2010).

29 Krippner-Heidenreich, A. et al. Single-chain TNF, a TNF derivative with enhanced stability and antitumoral activity. Journal of immunology 180, 8176-8183 (2008).

30 Debets, M. F., van Hest, J. C. & Rutjes, F. P. Bioorthogonal labelling of biomolecules: new functional handles and ligation methods. Organic & biomolecular chemistry 11, 6439-6455, doi:10.1039/c3ob41329b (2013).

31 Kiick, K. L., Saxon, E., Tirrell, D. A. & Bertozzi, C. R. Incorporation of azides into recombinant proteins for chemoselective modification by the Staudinger ligation. Proceedings of the National Academy of Sciences of the United States of America 99, 19-24, doi:10.1073/pnas.012583299 (2002).

32 Dixon, H. B. & Perham, R. N. Reversible blocking of amino groups with citraconic anhydride. The Biochemical journal 109, 312-314 (1968).

1. A method of modifying the N-terminus or the C-terminus of a peptide, polypeptide, or protein, comprising the steps of:
(a) incubating a derivative of the peptide, polypeptide, or protein in the presence of a reversible amine group blocking agent so that all amine groups in the derivative are blocked or in the presence of a reversible carboxyl group blocking agent so that all carboxyl groups in the derivative are blocked, wherein the derivative comprises an amino acid tag and a protease cleavage site appended to the N-terminus or the C-terminus of the peptide, polypeptide, or protein, such that the protease cleavage site is interposed between the amino acid tag and the N-terminus or the C-terminus of the peptide, polypeptide, or protein;
(b) contacting the blocked derivative with a protease that specifically cleaves at the protease cleavage site whereby the blocked derivative is cleaved;
(c) incubating the cleaved derivative with an amine reactive form of a reagent in a reaction mixture, whereby the N-terminus of the cleaved derivative is modified with the reagent to form a reagent-esterified, cleaved derivative or incubating the cleaved derivative with a carboxyl reactive form of a reagent in a reaction mixture, whereby the C-terminus of the cleaved derivative is modified with the reagent to form a reagent-esterified, cleaved derivative;
(d) removing the blocking groups from the amine groups or from the carboxyl groups in the reagent-esterified cleaved derivative.

2. The method of claim 1 wherein the derivative is made by expression of a recombinant DNA construct in a cellular or organismal expression system.

3. The method of claim 1 wherein the reagent is selected from the group consisting of: a peptide, a polypeptide, a cytotoxic agent, a ligand which specifically binds to a receptor, an antibody, an antibody fragment, a cytokine, a growth factor, a blood clotting factor, an imaging contrast agent, a radionuclide, a fluorescent moiety, a biopolymer, polyethylene glycol, β-Cyclodextrin caproate, and β-Cyclodextrin amino dodecanoate.

4. The method of claim 1 wherein the protease is Tobacco Etch Virus nuclear-inclusion-a endopeptidase (TEV).

5. The method of claim 1 wherein the reversible amine group blocking agent is pH sensitive.

6. The method of claim 5 wherein reversible amine group blocking agent is citraconic anhydride.

7. The method of claim 5 wherein the step of incubating the derivative is performed at basic pH.

8. The method of claim 5 wherein the step of incubating the cleaved derivative is performed at acidic pH.

9. The method of claim 1 wherein the peptide, polypeptide, or protein is selected from the group consisting of an antibody, an antibody fragment, a cytotoxic agent, TNF-α, a cytokine, a growth factor, and a blood clotting factor.

10. A preparation of a bioactive peptide, polypeptide, or protein that is modified at its N-terminus or its C-terminus by esterification with a reagent, wherein the preparation is homogeneous in the location of the esterification on the peptide, polypeptide, or protein, and wherein the bioactivity of the modified peptide, polypeptide, or protein is equivalent to the bioactivity of the peptide, polypeptide, or protein without modification.

11. The preparation of claim 10 wherein modification of the peptide, polypeptide, or protein with the reagent improves at least one of the properties selected from the group consisting of serum stability, pharmacokinetic properties, biodistribution, renal clearance, systemic toxicity, molecular or cellular targeting, and imaging contrast.

12. The preparation of claim 10 wherein modification of the peptide, polypeptide, or protein with the reagent imparts an additional bioactivity to the peptide, polypeptide, or protein.

13. A kit for modifying a peptide, polypeptide, or protein, comprising (a) a reversible amine blocking agent or a reversible carboxyl blocking agent; and (b) a protease.

14. The kit of claim 13 which comprises a reversible amine blocking agent and further comprises (c) a first buffer suitable for the reversible amine blocking agent to block free amine groups; and (d) a second buffer suitable for removal of blocking groups from the amine groups.

15. The kit of claim 14 wherein the first buffer is basic and the second buffer is acidic.

16. The kit of claim 13 which comprises a reversible carboxyl blocking agent and further comprises (c) a first buffer suitable for the reversible carboxyl blocking agent to block free carboxyl groups; and (d) a second buffer suitable for removal of blocking groups from the carboxyl groups.

17. The method of claim 1 which is for modifying the C-terminus of a peptide, polypeptide, or protein, and comprises:
(a) incubating a derivative of the peptide, polypeptide, or protein in the presence of a reversible carboxyl group blocking agent so that all carboxyl groups in the derivative are blocked, wherein the derivative comprises an amino acid tag and a protease cleavage site appended to the C-terminus of the peptide, polypeptide, or protein, such that the protease cleavage site is interposed between the amino acid tag and the C-terminus of the peptide, polypeptide, or protein;
(b) contacting the blocked derivative with a protease that specifically cleaves at the protease cleavage site whereby the blocked derivative is cleaved;
(c) incubating the cleaved derivative with an carboxyl reactive form of a reagent in a reaction mixture, whereby the C-terminus of the cleaved derivative is modified with the reagent to form a reagent-esterified, cleaved derivative;
(d) removing the blocking groups from the carboxyl groups in the reagent-esterified cleaved derivative.

18. The method of claim 1 which is for modifying the N-terminus of a peptide, polypeptide, or protein, and comprises:
(a) incubating a derivative of the peptide, polypeptide, or protein in the presence of a reversible amine group blocking agent so that all amine groups in the derivative are blocked or, wherein the derivative comprises an amino acid tag and a protease cleavage site appended to the N-terminus or the C-terminus of the peptide, polypeptide, or protein, such that the protease cleavage site is interposed between the amino acid tag and the N-terminus or the C-terminus of the peptide, polypeptide, or protein;
(b) contacting the blocked derivative with a protease that specifically cleaves at the protease cleavage site whereby the blocked derivative is cleaved;
(c) incubating the cleaved derivative with an amine reactive form of a reagent in a reaction mixture, whereby the N-terminus of the cleaved derivative is modified with the reagent to form a reagent-esterified, cleaved derivative;
(d) removing the blocking groups from the amine groups in the reagent-esterified cleaved derivative.
